(19) United States
(12) Patent Application Publication
(10) Pub. No.: US 2006/0263410 A1
(43) Pub. Date: Nov. 23, 2006

(54) Biological method for detoxication of a liquid food medium

(76) Inventors: Emmanuel Kossi Tangni, Louvain-La-Neuve (BE); Julien Simonis, Bruxelles (BE); Yvan Larondelle, Bruxelles (BE); Ludovic De Meeus D'Argenteuil, Chastre (BE)

Correspondence Address: CLARK & BRODY, 1090 VERMONT AVENUE, NW, SUITE 250, WASHINGTON, DC, 20005, US

(21) Appl. No.: 10/564,525

(22) PCT Filed: Jul. 15, 2004
(86) PCT No.: PCT/FR04/01863

§ 371 Date: Jun. 25, 2006

(30) Foreign Application Priority Data
Jul. 16, 2003 (FR) 0308689

(51) Int. Cl. C12C 11/00; A61K 47/00

(52) U.S. Cl. 424/439; 426/16

(57) ABSTRACT

The ivention concerns a biological method for decontaminating mycotoxins present in a liquid food product by adsorbing them onto insoluble vegetable fibers, and a brewing method comprising at least one decontamination step according to said method, as well as food products at least partly detoxified obtained by implementing said method.

The present invention relates to a biological process for decontaminating mycotoxins which are present in a liquid dietary product by absorbing the mycotoxins on insoluble plant fibers, to a brewing process which comprises at least one step of decontamination in accordance with this process, and to the at least partially detoxified dietary products which can be obtained by implementing such a process.

The importance of the innocuousness of foodstuffs for dietary safety has been widely recognized, in particular by the various governments which participated in 1992 in the International Conference on Nutrition which took place in Rome (Italy) and participated in 1996 in the Rome (Italy) World Food Summit. The quality and safety of foodstuffs can be threatened by a large number of factors, including by the presence of natural toxins.

Thus, in the long list of toxins which can naturally occur in everyday dietary products, the mycotoxins represent a very important category which is one of those which has been studied the most insofar as their ubiquity and their harmful effects on human and animal health give rise to general concern (FAO, 1999, “Preventing mycotoxin contamination”, publication No. 23, Rome, Italy, p. 55).

Agricultural products are the potential targets of pests and diseases. They carry a variable and numerous microbial flora which principally comprises bacteria, yeasts and filamentous fungi. The presence of these organisms can, in particular, give rise to deterioration in the quality of the agricultural products, sometimes amounting to their outright destruction.

Among these microorganisms, the filamentous fungi are responsible for producing the mycotoxins, as can be seen during the growth of the agricultural products in the field or else during their storage under favorable conditions of humidity and temperature. The main genera of mycotoxin-producing fungi are Penicillium, Fusarium, Aspergillus and Alternaria.

The mycotoxins are secondary metabolites whose chemical composition varies greatly but which are in general of low molecular weight. Their harmful effects, whether acute or chronic, on human health are also very varied. Their main targets are the kidneys, the liver, the gastrointestinal tract and the nervous and immune systems. About five hundred mycotoxins have been discovered to date and their number continues to increase as research advances. However, only about twenty have been well identified as being a real threat to dietary safety. Those different mycotoxin families encountered in dietary products which may be mentioned in particular are aflatoxins (AFLA), composed of aflatoxins B₁, B₂, G₁ and G₂, alternariol, fumonisin (FB), ochratoxin A (OTA), patulin (PAT) and trichothecenes, including vomitoxin, sterigmatocystin and zearalenone (ZEA). These mycotoxins can have a variety of harmful effects on human or animal health depending on their nature; they can, in particular, be hepatotoxic and immunotoxic, carcinogenic, teratogenic, neurotoxic or nephrotoxic or else lead to digestive disturbances or hemorrhages.

The main dietary foodstuffs which can be contaminated with mycotoxins are cereals, nuts, dry fruit, coffee, coco, spices, oilseeds, peas and broad beans as well as fruit. Their derived products can therefore be contaminated depending on the stability of the toxin during the transformation process. The result of this is that these mycotoxins, in particular OTA, can be transmitted to a large number of products of everyday consumption such as wine, beer, bread and products derived from coffee and cocoa (Abarca M L. et al., J. Food Prot., 2001, 64(6), 903-906; Walker R., Adv. Exp. Med. Biol. 2002, 504, 249-255). There is also a significant risk of secondary contamination by way of different foodstuffs of animal origin such as meat, milk, eggs and cheese (Pittet A., Revue Méd. Vét., 1998, 149, 479-492).

The contamination by mycotoxins of liquid dietary products which are prepared from grain or fruit is a growing subject of concern for manufacturers in the agro-industrial sector and, in particular, for brewers. Thus, improvement in detection and quantification instruments has made it possible to highlight the presence of a variety of mycotoxins in beer (see, in particular, Scott P. M. et al., Food Addit. Contam., 1995, 12(4), 591-598; Scott P. M., J. AOAC Int., 1996, 79(4), 875-882 and Scott P. M. et al., J AOAC Int., 1997, 80(6), 1229-34). The mycotoxins are broken down to some degree during the brewing process, but the data are variable and incomplete; complete disappearance of the toxins is rarely achieved. A large number of studies have reported the presence of mycotoxins, in particular OTA, in beer (see, in particular, the article by Nakajima M. et al., J. AOAC Int., 1999, 82, 897-902). The concentrations which are found are generally low and often below the proposed limit of 0.2 μg of OTA/l of beer. However, high concentrations are sometimes detected and regular consumption can result in the acceptable daily dose (ADD) being exceeded (Stettner G., 2001, “Nachweis und Verhalten von Deoxynivalenol und Ochratoxin A während der Bierbereitung [Detection and behavior of deoxynivalenol and ochratoxin A during beer preparation]”. Lehrstuhl für Technologie der Brauerei II [II chair of brewing technology], Munich Technical University, Germany). Furthermore, a number of governments, having become evermore aware of the problems linked to mycotoxins, are on the point of legislating on this matter.

There are also a very large number of articles dealing with this problem at the global level, of which those by Wolf-Hall C. E. et al., Adv. Exp. Med. Biol., 2002, 504, 217-226; Tangni E. K. et al., Food Add. Contam., 2002, 19(12), 1169-1179 and Odhav B. et al., Food Add. Contam., 2002, 19(1), 55-61 may in particular be mentioned.

The mycotoxins are compounds which are very stable and resistant to the majority of the processes for transforming agro-industrial products. Consequently, and taking into account their harmful effects on health, it is of the greatest importance to have available effective means for:
either preventing contamination of the dietary products,
or decontaminating the products before and/or after they have been transformed.

The first approach is not always feasible in view of the conditions for growing and storing the raw materials. The second approach should therefore be implemented at the industrial level, and a variety of physicochemical or biological processes have already been proposed with this aim in mind. These decontamination processes can be divided into two main categories:

1) the first category consists in breaking down the toxins into products which are less toxic or not toxic so as to ensure that ingesting them is less detrimental to the body;

2) the second category consists in using adsorbents in order to at least partly retain the mycotoxins. These adsorbents are used either during the manufacture of the dietary products, in order to ensure that the finished products intended for consumption contain the lowest possible quantity of residual mycotoxins, or are added to the consumed foodstuffs in order to reduce bioavailability. This elimination is generally effected using materials which are suitable for filtering or adsorbing the mycotoxins so as to reduce their availability.

One of the documents of the prior art describing filtration processes which may be mentioned, in particular, is the American patent U.S. Pat. No. 5,248,382, which describes a method for reducing the level of mycotoxins, in particular the level of patulin, in fruit juices by means of filtration through a microporous resin which is able to retain the patulin by chemisorption and whose pore diameter is less than 20 Angstrom. Although effective, this method suffers from the disadvantage of using a specific and expensive material.

In the large number of documents of the prior art which describe processes belonging to this second category, the mycotoxins are removed by the action of what are generally inorganic adsorbents, such as phyllosilicates such as clays and hydrated sodium calcium aluminosilicates (HSCAS), active charcoal and certain special polymers. Particular reference may be made, in this field, to the following:
the German patent application DE 3 810 004 relating to the use of bentonites;
the article by Arimoto-Kobayashi S. et al., Mutat. Res., 1997, 381(2), 243-249, which discloses the detoxifying ability, with regard to aflatoxin, of a mixed material based on chlorophyll, polyglucosamine and chitosan;
the International application WO 02/052950 relating to the use of a zeolite-based adsorbent powder containing more than 80% of a mixture of clinoptilotite and heulandite and a fatty-chain quaternary ammonium compound; and
the International application WO 02/40150, which describes the use of acid-activated lamellar silicates for adsorbing mycotoxins.

There are also methods which make use of biological decontamination processes. In this field, mention may be made, in particular, of the International application WO 98/34503, which describes a method for treating biological products which are contaminated with mycotoxins in which the contaminated product is brought into contact with lactic acid bacteria or propionic acid bacteria.

However, these processes taken together cannot always be used for detoxifying dietary products at the industrial level insofar as they are not in complete accord with the constraints of cost, of preserving the properties of the foodstuff and of harmlessness of the breakdown products which are generated during these processes. Furthermore, the adsorbents which are currently available on the market can give rise to a certain loss of nutrients in the final dietary product or else to poor metabolic utilization of these nutrients.

There is therefore a real need for developing a detoxification process which is directly applicable to the liquid dietary products which are especially derived from grain or fruit, in particular to the brewing process.

The inventors have set themselves the object of providing a biological process for detoxifying liquid dietary media, which process remedies all these drawbacks and can be applied simply and directly to detoxifying dietary products, in particular beer.

To this end, the inventors have surprisingly shown that adsorbing the mycotoxins on insoluble plant fibers makes it possible to decontaminate liquid dietary media and, as a consequence, to remove to a significant degree the mycotoxins which are likely to be present in the dietary products which are derived from these media.

The present invention is based on this phenomenon.

The present invention therefore relates to a biological process for decontaminating mycotoxins in a liquid dietary medium, characterized in that it comprises at least the following steps:
adsorbing at least a part of the mycotoxins, which are likely to be present in the liquid dietary medium to be decontaminated, by bringing said medium into contact with insoluble plant fibers, and
removing said fibers on which the mycotoxins are absorbed.

The biological process according to the present invention is based on the adsorbent effect of the insoluble plant fibers vis-á-vis the mycotoxins. It provides a particularly effective and simple solution, to be implemented at less cost, for decontaminating liquid dietary products of mycotoxins, with this generally being the case without any major modification of the manufacturing processes which are usually employed. In addition, it can advantageously be used directly during the brewing process, for which it exhibits the particular advantages of facilitating the filtration step and improving the stability of the foam.

According to one advantageous embodiment of the process according to the invention, the insoluble plant fibers are selected from the fibers derived from:
dietary plants such as cereals, leguminosae, culinary plants and fruits including tropical fruit, and, more generally, any plant which is used for nutritional purposes,
plants which are used by the paper industry, such as trees, sugarcane, bamboo and cereal straw.

The cereal-derived fibers which may in particular be mentioned are wheat, barley, oat, corn, millet, rice, rye and sorghum fibers and their malted equivalents.

According to the invention, a malted equivalent is understood as meaning germinated grain whose germination has been stopped by a heat treatment and which has then been freed of its germ material.

The dietary plant-derived fibers other than cereals which may in particular be mentioned are the fibers derived from apples, pears, grape berries, lupin and soya bean seeds, tomatoes, peas, coffee, etc.

The cereal fibers which are very particularly preferred are:
the wheat fiber isolates which are sold under the commercial designation Adfimax® 95, in particular Adfimax® 95 “y” and Adfimax® 95;
the micronized wheat fibers sold under the commercial designations Adfimax® 48 and Adfimax® BW;
the micronized barley fibers sold under the commercial designation Adfimax® 76 and, in particular, Adfimax® 76 and Adfimax® 76 “m” (medium);
the micronized oat fibers sold under the commercial designation Adfimax® 82;
the micronized apple fibers sold under the commercial designation Adfimax® 75;
the micronized grape fibers sold under the commercial designations Adfimax® 59 (pulp fibers) and Adfimax® 64 (pip fibers);
the micronized pea fibers sold under the commercial designations Adfimax® 90 and Adfimax® 56;
the micronized lupin fibers sold under the commercial designation Adfimax® 84;
the micronized soya fibers sold under the commercial designation Adfimax® 80;
   with all these fibers being available from the REALDYME company (28700 Garancièrcs en Beauce, France).

According to an advantageous and expedient embodiment of the invention, the plant fiber(s) which is/are employed are selected in dependence on the nature of the liquid dietary media to be decontaminated, that is to say from the fibers of the same origin as that of the products which make up the basic composition of the media to be decontaminated. Preference can, for example, be given to using apple fibers for decontaminating apple juices or else barley fibers for decontaminating beer.

The nature of the fibers which are employed in accordance with the process of the invention can also be selected in dependence on the nature of the mycotoxin(s) which is/are likely to be present in the liquid dietary medium to be decontaminated.

Thus, as far as the adsorption of OTA is concerned, preference is given to using the products Adfimax® 95, Adfimax® 82, Adfimax® BW or Adfimax® 75, or their mixtures.

As far as the absorption of deoxynivalenol (DON) is concerned, preference is given to using Adfimax® 95, Adfimax® 48 or Adfimax® 90 or their mixtures.

As far as FB is concerned, preference is given to using Adfimax® 82.

As far as the adsorption of aflatoxins, in particular aflatoxin B1 (AFB1) is concerned, preference is given to using Adfimax® 82.

According to one particularly advantageous embodiment of the process according to the invention, the plant fibers are preferably selected from micronized fibers. Thus, the inventors observed that using micronized plant fibers significantly increased the quantity of mycotoxin adsorbed per gram of fiber as compared with the quantity of mycotoxin which was adsorbed on nonmicronized plant fibers.

Micronization is a process which reduces the size of the particles. Thus, according to this particular embodiment, the plant fibers are then preferably present in the form of microparticles at least 90% of the total mass of which has a size of less than or equal to 700 μm and, even more preferably, at least 90% of the total mass of which has a size of less than or equal to 200 μm, with the granulometry being measured by sieving through an A 200 LS air jet sieve, which is marketed by the Alpine company (Augsburg, Germany). Fibers of this nature can, in particular, be prepared using the process described in the patent application FR 2 433 910.

According to the invention, preference is very particularly given to using fibers such as Adfimax® BW, which are micronized wheat fibers in the form of microparticles at least 90% of the total mass of which has a size of less than or equal to 200 μm.

In addition, and in order to avoid any absorption of the medium by the fibers in connection with the latter being brought into contact with the liquid dietary medium to be decontaminated, the process according to the invention also preferably comprises a preliminary step during which the fibers are hydrated. This preliminary hydration of the fibers does not significantly affect their potential for adsorption vis-à-vis the mycotoxins.

According to the process in accordance with the invention, the quantity of plant fibers which is introduced into the liquid medium to be decontaminated is preferably between 0.1 and 20%, and even more preferably between 0.5 and 5%, with these percentages being expressed in weight of fibers (before any possible hydration) per liter of medium.

The dietary medium is preferably brought into contact with the plant fibers for a period which can vary between a few seconds and 90 minutes, more preferably between 5 and 45 minutes. Thus, the inventors have observed that the adsorption takes place very rapidly and is irreversible for at least 48 hours at constant temperature.

While the pH of the liquid dietary medium is not critical in accordance with the invention, the bringing into contact with the plant fibers is preferably effected at an acid pH of between 1.5 and 7. When the process according to the invention is integrated with a brewing process, the pH of the liquid dietary medium is preferably between 5.2 and 5.4.

Moreover, as far as the adsorption of OTA is concerned, the inventors have surprisingly shown that the percentage of mycotoxins which is extracted from the medium for a given quantity of insoluble plant fibers, in particular Adfimax® BW fibers, is multiplied by approximately two when the pH of the medium changes from a value of approximately 6 to a value of approximately 2.5.

The pH of the medium can naturally be adjusted to the desired value using alkalinizing or acidifying agents such as those which are customarily used in the food processing industry.

Even though it is no longer critical, the temperature of the medium to be decontaminated can also have an influence on the quantity of mycotoxins adsorbed for a given quantity of fibers, with this quantity generally tending to decrease as the temperature increases. Thus, according to one advantageous embodiment of the process according to the invention, the medium is maintained at a temperature of between approximately 7 and 80° C., preferably between approximately 20 and 30° C., during the whole of the period of contact.

The different liquid dietary media which can be decontaminated in accordance with the process according to the invention and which may be mentioned in particular are beer, mixtures of malt and water and the mash of the brewing processes, wine, coffee, fruit juices, milk and glucose syrups.

At the end of the period of contact, the fibers are preferably removed by filtration using the techniques which are known to, and customarily used by, the skilled person for this purpose.

According to one variant of the invention, the steps of bringing the liquid dietary medium to be decontaminated into contact with the insoluble plant fibers, on the one hand, and, on the other hand, of removing said fibers on which the mycotoxins are adsorbed, can be carried out simultaneously. In this case, the step of removing the fibers is generally a step of filtration and the insoluble plant fibers can then, for example, form an integral part of a filtration system and, in particular, be present in the form of a filtration adjuvant.

The decontamination process in accordance with the invention, and as has just been described above, enjoys the advantage of being able to be directly integrated into the brewing process without it being necessary to significantly modify the steps of this process.

In principle, this process involves three ingredients which are barley malt, water and hops. While the production process can be carried out in a variety of ways which are well known to the skilled person, it is generally possible to distinguish a few main steps which are:
malting, which comprises germinating the previously soaked grain,
masking in the strict sense of the word, which represents the dissolution of the soluble matter of the malt, which matter has already been partially broken down by the germination, and of the raw grain, with this resulting in a mash,
filtration of the mash,
boiling: the filtrate (also termed wort) is subsequently brought to boiling for varying periods and then cooled down again,
fermentation of the wort and its inoculation with yeast,
standing: this is the time for the maturation of the beer, with the time varying in dependence on the beer,
filtration: after its period of standing, the beer is once again filtered before being drawn off and packaged.

As a result, the invention therefore also relates to the use of the mycotoxin decontamination process according to the invention for detoxifying beer during a brewing process, with said brewing process involving at least one filtration operation.

This particular application of the process in accordance with the invention not only makes it possible to decontaminate beer of mycotoxins but also enjoys the following additional advantages:
of improving the quality of the filtrations, in particular that of the mash, taking into account the increase in the content of fibers in the filtration cake;
of improving the stability of the foam, and
of facilitating the clarification of the wort during the filtration and the clarification.

The invention therefore relates to a brewing process which comprises at least one step of mashing and at least one step of fermenting a wort, characterized in that it additionally comprises at least one step of mycotoxin decontamination in accordance with the previously described process, with said decontamination step taking place simultaneously with the mashing step and/or after the step of fermenting and, where appropriate, of maturing the wort.

According to a first embodiment of this process, the decontamination step is carried out simultaneously with the mashing step by bringing a mixture of ground malt and water into contact with insoluble, preferably hydrated, plant fibers, with said fibers on which the mycotoxins are now adsorbed being removed by the step of filtering the mash.

In this case, the plant fibers are preferably introduced at the rate of approximately from 0.5 to 20% by weight based on the weight of malt.

According to a second embodiment of this process, the step of bringing the liquid medium to be decontaminated into contact is carried out before the step of filtering a wort which is fermented and, where appropriate, matured, by bringing this wort into contact with insoluble, preferably hydrated, plant fibers, with said fibers on which the mycotoxins are now absorbed, being removed by the step of filtering the fermented wort (beer).

In this case, the plant fibers are preferably introduced into the fermented wort at the rate of approximately from 0.05 to 5% by weight based on the total weight of the wort.

Finally, the invention relates to the dietary products which are at least partially decontaminated of mycotoxins and which can be obtained by implementing the decontamination process according to the invention and, more specifically, to decontaminated liquid dietary products such as beer and fruit juices as well as to decontaminated solid dietary products such as lyophilized powders, for example coffee, or else dairy products such as yoghurts and cheeses.

In addition to the abovementioned measures, the invention also comprises other measures which will emerge from the description which follows and which refers to an example demonstrating the OTA-adsorption properties of the insoluble plant fibers and preliminary screening of different plant fibers, to an example demonstrating the adsorption of OTA in a brewing wort, to an example of studying the effect of the temperature on the adsorption of OTA in a model liquid medium, to a study of the impact of adding insoluble vegetable fibers on mixing in a micromashtub, to a study of the effect of the pH on the adsorption of the mycotoxins by wheat fibers, to an example demonstrating the adsorption of OTA on plant fibers in grape juice, to an example demonstrating the adsorption of B1 aflatoxins by insoluble plant fibers, to a study of the effect of micronization of different insoluble plant fibers on the quantity of B1 aflatoxin adsorbed, to a study of the dose-response effect of the adsorption of aflatoxin B1 by wheat fibers which are or are not micronized, to an example concerning the adsorption of the OTA during a pilot brewing process, and to an example of the adsorption of OTA by grape fibers in a model medium, as well as to the attached FIGS. 1 to 8 in which:

FIG. 1 depicts the change in the adsorption of OTA by five different fibers (Adfimax® 95 y; Adfimax® 95; Adfimax® BW; Adfimax® 76 and Adfimax® 76 m) in a model liquid medium containing an initial concentration of 30 ng of OTA/ml, for an initial volume of 25 ml and a contact period of 45 minutes, as a function of the quantity of fibers in grams per liter of medium;

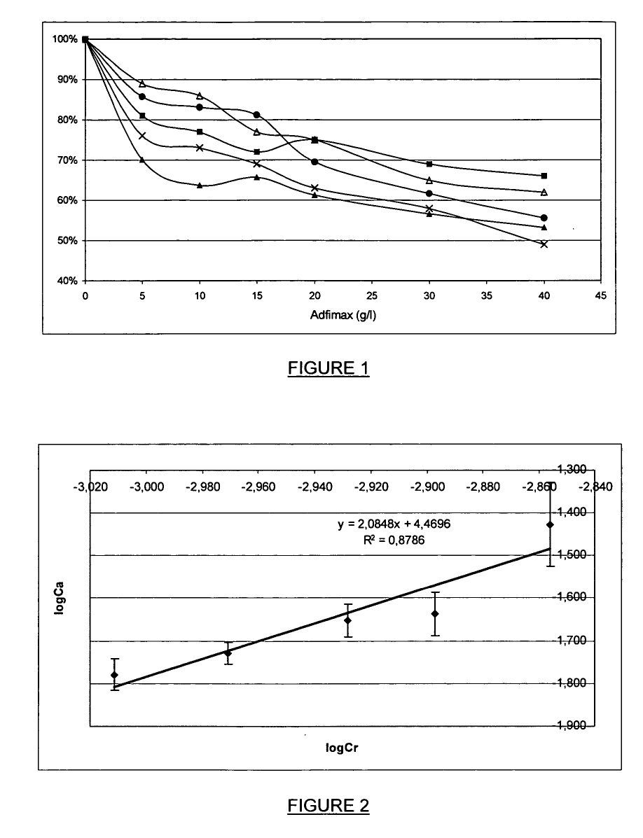

FIG. 2 depicts the modeling of the adsorption phenomenon in accordance with Freundlich's theory, that is the log of the concentration of OTA adsorbed by Adfimax® BW as a function of the log of the concentration of residual OTA in the supernatant. This makes it possible to estimate the capacity for adsorbing the OTA and the affinity of Adfimax® BW at a temperature of 25° C.;

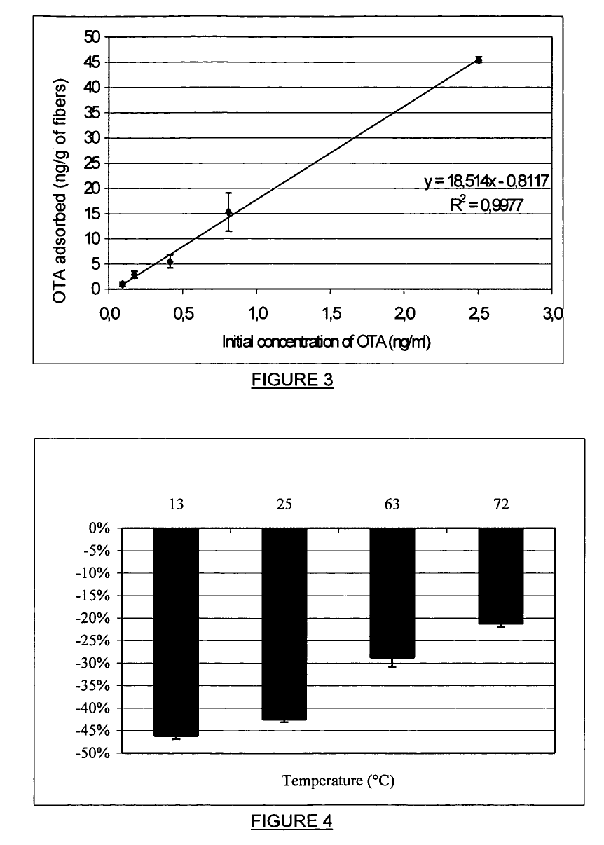

FIG. 3 depicts the quantity of OTA (ng) which is adsorbed per gram of Adfimax® BW fibers as a function of the initial concentration of OTA in the medium in ng/ml;

FIG. 4 depicts the effect of the temperature on the adsorption of OTA by Adfimax® BW fibers; in this figure, the black bars represent the decrease in the concentration of OTA (in %) in the medium during direct contact, at different temperatures, of the fibers with the contaminated medium;

FIG. 5 depicts the quantity of filtrate recovered (ml) as a function of the time (minutes) for different doses of Adfimax® BW which were added to a mixture of malt and water during a brewing process;

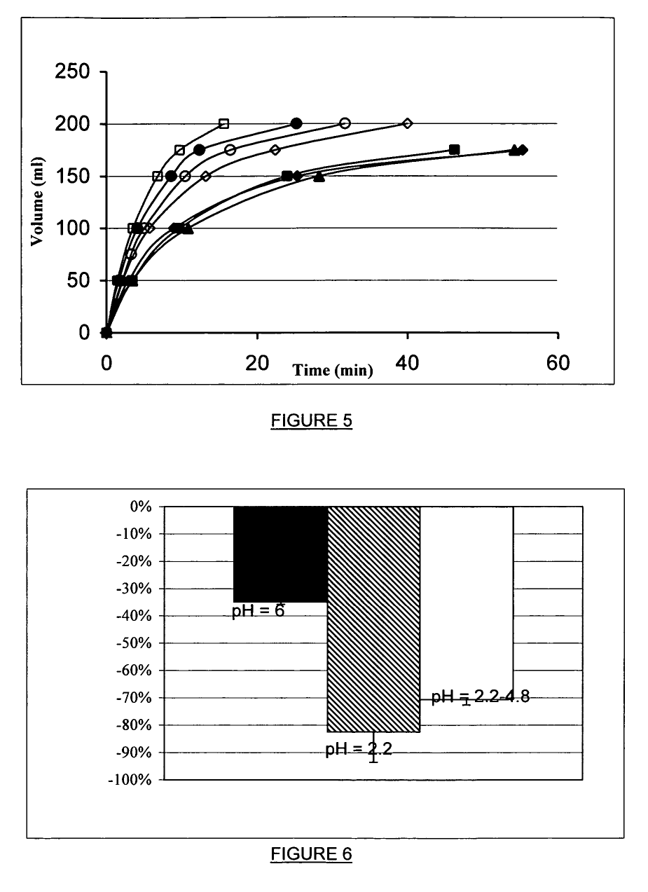

FIG. 6 depicts the change, in a model liquid medium, in the adsorption of OTA (decrease in the quantity of OTA as a percentage of the quantity initially present) by Adfimax® BW fibers as a function of changes in the pH;

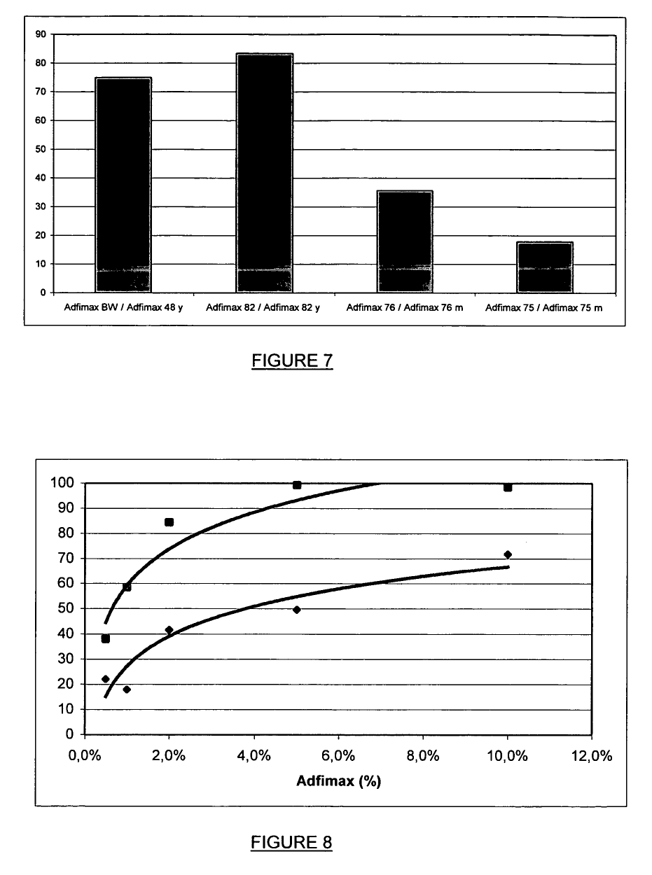

FIG. 7 depicts the difference (in %) between the micronized, nonmicronized and medium forms of fibers of the same origin in the quantity of aflatoxin B1 adsorbed (in ppb) and shows that the quantity of mycotoxin adsorbed is linked to the size of the particles;

FIG. 8 depicts the quantity of AFB1 adsorbed, expressed as a percentage of the quantity of AFB1 which was initially present in a model medium, as a function of the quantity of fibers employed (in % by weight); (black squares: micronized wheat fibers and black diamonds: nonmicronized wheat fibers).

### EXAMPLE 1

### Demonstration of the OTA-Adsorbing Properties of the Insoluble Plant Fibers and Preliminary Screening of Different Plant Fibers

The inventors have surprisingly demonstrated that incorporating plant fibers into a model liquid medium makes it possible, as a result of the OTA being adsorbed on the fibers, to decrease the quantity of available toxin in this medium. The in-vitro tests which are reported in this example were carried out in order to demonstrate the adsorption properties of the micronized plant fibers when they are incorporated into a liquid medium which is contaminated with OTA and in order to determine the contact time which is required for the OTA to be adsorbed optimally by these fibers. Subsequently, five different plant fibers were screened in order to determine the fibers which were most efficient with regard to adsorbing the OTA. Each of the micronized fiber types which was used was tested against the corresponding nonmicronized fibers.

1) Experimental Protocol

A predetermined quantity of plant fibers (approximately 20 g/l) is mixed, in a sterile 50 ml tube, with 25 ml of an aqueous solution which contains 2% glucose (sold by Merck under the commercial name D(+) glucose monohydrate), 5% yeast extract (sold by ICN Biomedical under the commercial name powdered yeast extract) and 1% peptone (sold by Duchefa under the name peptone) and which was previously sterilized at 121° C. for 15 minutes. This aqueous solution has a pH of between 6.0 and 6.2 and is designated “DYP” in that which follows (model medium). The DYP solution is then contaminated with a variable quantity of a solution of OTA in ethanol. The concentration of OTA in the model liquid medium is 57 ng/ml. The contents of the tube are then homogenized by shaking manually for 30 seconds, after which the tube is placed to be stirred at 90 revolutions per minute (rpm) for 45 minutes in a room which is thermostated at 25° C. A control treatment (control) without adsorbent, that is to say without plant fiber, is carried out in the case of each experiment, and each of these experiments is performed three times.

The suspension is then centrifuged at 1830 g for 10 minutes at a temperature of 25° C., after which the pellet is separated from the supernatant. 1 ml of supernatant is then extracted, in a sterile tube, with 9 ml of a solution of methanol:water (50:50; v/v). The tube is then vortexed for 30 seconds, after which it is centrifuged for 10 minutes at 820 g at a temperature of between 5 and 10° C. This extract is then diluted and filtered and analyzed by high performance liquid chromatography (HPLC).

The HPLC system consists of a Perkin Elmer® LC049 isocratic pump, sold by Norwalk (USA), together with a 50 μl injection loop sold by Cotati (USA) under the name Rheodyne®, equipped with a C₁₈ column of 150 mm in length and 4 mm in diameter, sold under the name Hypersil® BDS, of 3 μm porosity, sold by Tracer Analytica (Spain), with an RF 551 fluorescence detector sold by Shimadzu (Japan) fitted with a xenon lamp of 150 W intensity adjusted to an excitation wavelength (λ_{excitation}) of 332 nm and an emission wavelength (λₑₘᵢₛₛᵢₒₙ) of 462 nm, and with an SP4290 integrator sold by Spectra Physics (USA). The mobile phase is composed of an acetonitrile/water/acetic acid (450/540/10; v/v) mixture which is filtered through a 0.25 μm membrane and degassed with helium for 15 minutes. The flow rate of the liquid phase is set at 1 ml/min at a pressure of between 2900 and 3000 psi.

The total quantity of OTA adsorbed is given by the difference between the initial quantity and the final quantity present in the supernatant.

The following fibers were used in this example: Adfimax® 95 y, Adfimax® 95, Adfimax® BW, Adfimax® 76 and Adfimax® 76 m, with each type of fiber being used at different doses.

2) Results

The results which were obtained are reported in Tables I to IV below and in the attached FIG. 1.

The percentages of OTA which were adsorbed on Adfimax® BW in dependence on the duration of the contact, when using the model medium containing 57 μg of OTA/l and at a pH of between 6.0 and 6.2, are reported in Table 1 below:

TABLE I

| Quantity of | Decrease in the concentration of OTA (%) | | |
|---|---|---|---|
| Adfimax ® BW | Duration of the period of contact (hours) | | |
| (g/l) | 3 | 24 | 48 |
| 0 (control) | 0 | 0 | 0 |
| 10 | 46.7 | 52.5 | 49.8 |
| 16 | 59.8 | 65.3 | 61.6 |
| 20 | 68.9 | 69.7 | 71.7 |
| 30 | 68.0 | 72.3 | 73.6 |

These results show that the adsorption of the fibers does not vary between 3 and 24 hours. Furthermore, the quantity of the OTA adsorbed increases in dependence on the quantity of fibers which are present in the medium.

The effects of periods of contact of less than 24 hours on the levels of OTA adsorption (in %) by the fibers (20 g of Adfimax® BW fibers per liter of model liquid medium which is at pH 5.2 and contains 35 ng of OTA/ml) are reported in Table II below:

TABLE II

| Duration of contact (in minutes) | % of OTA adsorbed |
|---|---|
| 0 | 0 |
| 5 | 21 |
| 15 | 20 |
| 45 | 25 |
| 90 | 29 |
| 169 | 28 |
| 360 | 30 |
| 1440 | 43 |

These results show that the adsorption takes place very rapidly (between about 5 and 45 minutes) and that this adsorption is maintained at least during the whole of the duration of the experiment.

The effects of micronization on the quantity of OTA adsorbed by Adfimax® BW fibers, and the quantities adsorbed by their nonmicronized starting material, are reported in Table III below:

TABLE III

Quantity of OTA adsorbed (%)

| | Nonmicronized | |
|---|---|---|
| Quantity of fibers (g/l) | starting material | Adfimax ® BW |
| 20 | 17% | 33% |
| 30 | 22% | 37% |

These results show that micronization improves the adsorption properties of the fibers.

The attached FIG. 1 depicts the quantities of OTA which are respectively adsorbed by five different fibers (Adfimax® 95 y: filled triangles; Adfimax® 95: filled squares; Adfimax® BW: crosses; Adfimax® 76: filled circles and Adfimax® 76 m: empty triangles) in DYP medium containing an initial concentration of 30 ng of OTA/ml, with an initial volume of 25 ml and a 45 minute duration of contact. In this figure, the percentage of residual OTA is expressed, in the case of each fiber, as a function of the quantity of fibers in grams per liter of DYP medium.

The results depicted in FIG. 1 show that a good adsorption of OTA, in particular when using Adfimax® 95 y, is observed even at fiber concentrations which are as low as 5 g per liter of medium.

### EXAMPLE 2

### Demonstration of the Adsorption of OTA in a Brewing Wort

A predetermined quantity of Adfimax® BW fibers (20 g/l) is mixed with 47 ml of clarified wort which is contaminated with 1.5 μg of OTA/l. The contents of the tube are then homogenized by shaking manually for 30 seconds, after which the tube is placed to be stirred at 90 revolutions per minute (rpm) for 45 minutes in a room which is thermostated at 25° C. A control treatment (control) without absorbent, that is to say without plant fiber, is also implemented in the case of each experiment in order to check for any possible spontaneous disappearance of OTA. Each assay is carried out in triplicate.

The suspension is then centrifuged at 1830 g for 10 minutes at a temperature of 25° C., after which the pellet is separated from the supernatant and extracted in the following manner: 20 ml of supernatant are diluted with 2.5 ml of water containing 4% by weight of sodium bicarbonate and 7.5 ml of phosphate buffer (PBS). The mixture is then centrifuged for 10 minutes at 820 g and at a temperature of between 5 and 10° C. 22.5 ml of the supernatant are then injected, at the rate of from 1 to 2 ml/minute, into an immunoaffinity column which is sold by Vicam under the name OchraTest®, with said column having been previously conditioned with 20 ml of a PBS solution. The column is then rinsed with 10 ml of water, after which it is eluted with methanol (2 ml) and then with water (2 ml). 20 ml of atmospheric air are then passed through the column in order to collect all the eluate, which is then filtered through a filter having a pore diameter of 0.45 μm and subsequently analyzed by HPLC using the protocol described above in Example 1.

The absorption values are then compared using Freundlich's empirical isotherm, which is given by the following equation (I):
Cₐ=k*Cᵣⁿ (I)
in which:
Cₐ is the quantity of OTA which is absorbed by unit weight of adsorbent (μg/g);
Cᵣ is the concentration of unabsorbed OTA at equilibrium (μg/ml);
k is a constant relating to the adsorption capacity of the adsorbent for OTA, and
n is a constant relating to the affinity of the adsorbent for OTA.

The linear regression curves were calculated between the logarithmic values of Cₐ and Cᵣ and a correlation coefficient R² was used for verifying the validity of the curve.

In order to assess the kinetics of the observed adsorption phenomenon, an experiment is performed during which a quantity of fibers corresponding to a proportion of 11 g/l is brought into contact with the wort which has previously been contaminated with increasing doses of OTA (from 0 to 2.5 ng/ml). The adsorption of the OTA is analyzed as described above.

2) Results

The results which were obtained are reported in Table IV below and in the attached FIGS. 2 and 3.

FIG. 2 depicts the Freundlich's isotherm of the log of the quantity of OTA adsorbed per unit weight of Adfimax® BW as a function of the log of the concentration of residual OTA in the supernatant. This makes it possible to estimate the capacity for adsorbing OTA, and the affinity of the Adfimax® BW at a temperature of 25° C.

FIG. 3 depicts the quantity of OTA (ng) which is adsorbed per gram of Adfimax® BW fibers as a function of the initial concentration of OTA, in ng/ml, in the medium.

TABLE IV

| Quantity of fibers (g/l) | Quantity of OTA adsorbed (%) |
|---|---|
| 1 | 6 ± 3 |
| 5 | 12 ± 3 |
| 15 | 22 ± 3 |
| 20 | 28 ± 2 |
| 30 | 35 ± 2 |
| 40 | 41 ± 4 |

These results show that the Adfimax® BW fibers are able to adsorb OTA in the brewing wort. They also show that the quantity of OTA adsorbed increases with the quantity of fibers present in the brewing wort. Furthermore, Freundlich's empirical model depicted in FIG. 2 corresponds to the experimental values for the adsorption of OTA by the Adfimax® BW fibers with a correlation coefficient R² of 0.8786. The adsorption capacity of the fibers, as calculated using Freundlich's adsorption constant, was 29.5 mg of OTA/g of fibers, and the affinity constant was 2.08 ml/g of fibers.

Finally, the results depicted in FIG. 3 show that the relationship between the quantity of OTA adsorbed per gram of fibers and the quantity of OTA which was initially present in the medium to be decontaminated is linear.

By way of comparison, phyllosilicates, diatomaceous earths, bentonite, HSCAS aluminosilicate and choleteramine, which compounds are known for their property of adsorbing mycotoxins, and which were tested under the same conditions, respectively gave the following adsorption capacities: from 0.3 to 0.44; from 0.5 to 1.5; 1.5-9.0; 2.2 and 9.6 mg/g. These values are lower than those obtained using the Adfimax® BW fibers.

### EXAMPLE 3

### Effect of the Temperature on the Adsorption of OTA in a Model Liquid Medium

According to the process in accordance with the invention, and as previously described, it has been pointed out that it is possible to decontaminate beer using insoluble plant fibers without making any major change in the brewing process and, in particular, at the same time as the mixing step, when the ground malt and the water are mixed. Given that this mixture is then brought to temperatures ranging up to 78° C., it is important to demonstrate that adsorption does indeed take place at these temperatures.

1) Experimental Protocol

The contaminated synthetic model medium DYP is treated beforehand in the same manner as in Example 1 above.

In order to study the effect of the temperature on the adsorption of OTA by the fibers, the contaminated DYP medium is brought to temperatures of 12, 25, 63 and 72° C. for 10 to 15 minutes before introducing the Adfimax® BW fibers. The DYP medium is then contacted with the fibers for 45 minutes at each of these temperatures in a thermostated water bath. The OTA which is adsorbed on the fibers at each temperature is quantified.

Separation, extraction, purification and analysis of the quantity of OTA are carried out in accordance with the protocol described above in Example 1.

2) Results

The results obtained, that is to say the effect of the temperature on the adsorption of OTA by Adfimax® BW fibers, are depicted in the attached FIG. 4.

In this figure, the black bars represent the adsorption of OTA (in %) during direct contact of the fibers with the contaminated medium at each of the temperatures tested (direct adsorption).

These results show that a rise in the temperature causes a decrease in the adsorption of the OTA on the fibers.

### EXAMPLE 4

### Study of the Impact of the Addition of Insoluble Plant Fibers on Mashing in a Micromashtub

The aim of this example is to verify that introducing insoluble plant fibers into the malt does not have any negative impact on the mashing.

1) Experimental Protocol

100 grams of malt are weighed in a cylinder whose tare is known and mixed in the cylinder with 350 ml of “reverse osmosis” (RO) water at 63° C. Different quantities of Adfimax® BW fibers, which have been previously saturated in RO, are added to different cylinders in order to obtain the fiber proportions of 0.5; 1; 2; 5; 10 and 15%. The contents of the cylinders are then carefully mixed, after which the cylinders are inserted into a water bath which is thermostated at 63° C., with each cylinder being surmounted by a stirrer. The temperature curve is as follows: 30 minutes at 63° C., 20 minutes at 72° C. and 1 minute at 78° C. The cylinders are then removed from the water bath, dried rapidly and weighed and their contents are passed through a cellulose filter which is resting on a 500 ml graduated base. The volume of the filtrate is noted in dependence on the time.

When the filtration has come to an end, the density of each of the resulting worts is measured, thereby making is possible to calculate the yield of extract (percentage of the soluble matter contained in the grain which was actually dissolved in the wort), as well as its color on a scale devised for this purpose by the “European Brewing Convention” (EBC).

In order to measure the fermentability of the wort (final attenuation), 100 ml of wort are then removed and mixed, under sterile conditions in a bottle, with 20 ml of a suspension of brewer's yeast.

In this example, each experiment is carried out in duplicate and identical measurements are also carried out on a wort which has not been brought into contact with the fibers, in order to serve as a control.

2) Results

The results which were obtained are reported in Table V below:

TABLE V

| Quantity of | | | | Final |
|---|---|---|---|---|
| fibers (% by | Density | Yield of | Color | attenuation |
| weight) | (g/100 g) | extract (%) | (EBC) | (%) |
| 0 (control) | 18.9 | 74.72 | 12 | 86.67 |
| 0.5 | 18.7 | 74.92 | 12 | 86.63 |
| 1 | 18.2 | 74.53 | 12 | 86.26 |
| 2 | 18.2 | 74.76 | 12 | 85.27 |
| 5 | 17.7 | 76.61 | 13 | 86.72 |
| 10 | 17.9 | 78.57 | 14 | 87.93 |
| 15 | 17.1 | 81.35 | 14.5 | 85.50 |

The attached FIG. 5 depicts the quantity of filtrate recovered, in ml, as a function of the time, in minutes, for the different doses of fibers which were added (control (0%): filled squares; 0.5%: filled diamonds; 1%: filled triangles; 2%: empty diamonds; 5%: empty circles; 10%: filled circles; 15% empty squares).

These results show that the addition of fibers is also greatly to the advantage of the filtration. Moreover, it is important to note that integrating the decontamination process in accordance with the present invention into a brewing process has no negative influence on the progress of the latter and, in particular, no negative influence on the fermentability of the wort (final attenuation).

### EXAMPLE 5

### Study of the Effect of the pH on the Adsorption of the Mycotoxins by Wheat Fibers

In order to study the impact of the pH, an experiment which consisted in measuring the adsorption before and after a decrease and then an increase in the pH was carried out in a model medium.

1) Experimental Protocol

A known quantity of Adfimax® BW fibers, corresponding to a concentration of 20 g of fiber/l, is mixed in a 50 ml sterile tube containing 25 ml of DYP model medium which is as described above in Example 1 and which has being contaminated beforehand with 50 ng of OTA/ml by means of adding a solution of OTA in ethanol. The pH of the medium is measured to be 6.

The contents of the tube are then incubated, separated, extracted, purified and analyzed as described above in Example 1.

In parallel, the pH of the same DYP medium is lowered, in two other tubes which are already provided with fibers, down to a value of 2.2 by adding solid lactic acid. The contents of one of the two tubes are then incubated, separated, extracted, purified and analyzed as described above in Example 1.

The pH of the medium in the second tube is then raised to 4.8 by adding sodium hydroxide granules. The contents of the tube are then incubated, separated, extracted, purified and analyzed as in Example 1.

Each experiment is carried out in triplicate.

2) Results

The results which were obtained are reported in the attached FIG. 6, which depicts the progress of the adsorption of OTA (decrease in the quantity of OTA in the DYP medium in % of the quantity which was initially present) on the fibers after the decrease, and after the increase, in the pH in the DYP medium. In this figure, the black bar corresponds to the measurements made at pH 6, the hatched bar corresponds to the measurements made at pH 2.2 and the white bar corresponds to the measurements which were made after the increase of the pH from 2.2 to 4.8.

It is evident that the adsorption of OTA by the fibers increases as the pH decreases, with a percentage adsorption of 82.4% being achieved at a pH of 2.2. The release of the toxin when the pH is raised does not appear to be as great as the increase in the adsorption when the pH is lowered.

For the same quantity of fibers, therefore, lowering the pH markedly increases the quantity of OTA which is adsorbed by these fibers.

### EXAMPLE 6

### Demonstration of the Adsorption of OTA on Plant Fibers in Grape Juice

1) Experimental Protocol

A weighed quantity of Adfimax® BW fibers is mixed with 25 ml of shop-bought grape juice which has been contaminated beforehand at the rate of 400 ng of OTA/l of grape juice using wort which is naturally contaminated with OTA. The weight of fibers is calculated so as to correspond to the concentrations of 20 and 50 g of fibers/l of grape juice.

The tubes are then mixed, purified, extracted and analyzed by HPLC as described above in Example 2.

A control treatment (without fiber) is performed in parallel and each of the treatments is carried out in triplicate.

2) Results

The results relating to the decrease in the quantity of OTA contained in the grape juice in dependence on the dose of fibers added are reported in Table VI below:

TABLE VI

| Dose of fiber (g/l) | Percentage adsorption |
|---|---|
| 20 | 74 ± 1.8% |
| 50 | 84.8 ± 2.4% |

These results show that bringing the grape juice into contact with these fibers leads to a good decrease in the concentration of OTA. The percentage adsorption is very high, with it being possible to attribute this in part to the acidity of the grape juice.

### EXAMPLE 7

### Demonstration of the Adsorption of B1 Aflatoxins by Insoluble Plant Fibers

A predetermined quantity of plant fibers (Adfimax® BW: 20 g/l) is introduced into a sterile 50 ml tube and mixed with 25 ml of pH 7 phosphate buffer (PBS) which has been previously contaminated with B1 aflatoxins (approximately 8.5 ppb). After having been homogenized manually for 30 seconds, the tube is placed to be stirred at 90 revolutions per minute for 45 minutes in a room which is thermostated at 25° C. A control treatment without adsorbent served as the control.

At the end of this period, the suspension is then centrifuged at 1830 g for 10 minutes and at 25° C., after which the pellet is separated from the supernatant. The assay is carried out in triplicate.

The (initial and residual) B1 aflatoxins are analyzed by a direct competitive ELISA immunochemical method using the high-sensitivity specific and quantitative test which is sold by Neogen Corporation (USA) under the trade name Veratox® HS. The protocol used was that recommended by the supplier of this test.

This immunochemical (ELISA) test was carried out in the following manner:
deposition of 100 μl of conjugate in each microwell, which is not coated with antibody;
addition of 100 μl of standard or of 100 μl of sample, and mixing;
withdrawal of all the mixture and its deposition in a microwell which is coated with antibody;
incubation for 10 minutes at room temperature;
washing five times with deionized water;
deposition of 100 μl of substrate;
incubation for 10 minutes at room temperature;
addition of 100 μl of the “Red Stop” solution which is provided with the test in order to stop the substrate-enzyme-reaction.

The same experiment is carried out in parallel at pH 3 in PBS medium (the pH of which has been adjusted to 3 with lactic acid) while a standard curve is also constructed using standards.

The optical densities of the colored solutions are then read at a wavelength of 620 nm using a microplate reader sold under the trade name Labsystem Multiscan MCC/340-RS232C (Labsystems, Finland).

The detection and quantification limits of this analytical method are respectively estimated to be 3 and 10 ppt while the percentage recovery is 100%.

The results of the assay are presented in Table VII below:

TABLE VII

| Duration of | B1 aflatoxins | B1 aflatoxins |
|---|---|---|
| contact (min) | adsorbed at pH 7 (%) | adsorbed at pH 3 (%) |
| 0 | 0 | 0 |
| 5 | 66 | 72 |
| 25 | 68 | 69 |
| 45 | 68 | 70 |
| 120 | 67 | 74 |

These results show that Adfimax® BW adsorbs a large quantity of B1 aflatoxins from 5 minutes of contact and upwards, both at neutral pH and at acid pH.

### EXAMPLE 8

### Study of the Impact of Micronization of Different Insoluble Plant Fibers on the Quantity of AFB1 Adsorbed

The aim of this example is to study in vitro the impact of the micronization of different insoluble plant fibers on the quantity of aflatoxin B1 (AFB1) absorbed.

The following fibers were used in this example:
nonmicronized (Adfimax® 48 y) and micronized (Adfimax® BW) wheat fibers,
oat chaff (Adfimax® 82 y) and micronized oat fibers (Adfimax® 82),
medium (Adfimax® 76 “m”) and micronized (Adfimax® 76) barley fibers,
medium (Adfimax® 75 “m”) and micronized (Adfimax® 75) apple fibers.

This study was carried out in a model medium consisting of 25 ml of PBS solution at pH =3 (25 ml per bottle), with each bottle being contaminated with approximately 8 ppb of AFB1. The plant fibers are the introduced into the contaminated medium at the rate of 20 g/l. Each bottle is stored for 45 minutes on a shaking table in a dark room at a temperature of 25° C. The bottles are then centrifuged at 3000 rpm and at 25° C. for 10 minutes. The supernatant is then recovered so as to stop the adsorption of the AFB1 by the fibers and the concentration of residual (that is unabsorbed) AFB1 is assayed in each of the supernatants using an ELISA test (“Veratox® for Aflatoxin HS”, sold by Neogen Corporation, USA). Each of these experiments is carried out in triplicate.

The results which were obtained are reported in the attached FIG. 7, which depicts the increase in the quantity of mycotoxin adsorbed by micronized fibers (in %) as compared with nonmicronized fibers of the same origin and by micronized fibers (in %) as compared with medium fibers of the same origin.

This figure shows that, within biological adsorbents, the micronization treatment has a very positive effect on adsorption. The figure also shows that the more comminuted the product is, the more it adsorbs.

In addition, it is observed that, of the micronized fibers, the micronized oat fibers adsorb all of the AFB1, thereby demonstrating the remarkable efficacy of the natural adsorbents in accordance with the invention.

### EXAMPLE 9

### Study of the Dose-Response Effect During the Adsorption of the AFB1 by Micronized or Nonmicronized Wheat Fibers

The aim of this example is to determine from what dose the micronized (Adfimax® BW) and nonmicronized (Adfimax® 48 y) wheat fibers adsorb the same quantity of AFB1.

The experiment was carried out in PBS buffer at pH 3 (with the pH having been adjusted with lactic acid). The PBS buffer was first of all contaminated with a content of approximately 8 ppb of AFB1.

The dose-response effect was evaluated for doses of 0.5%, 1%, 2%, 5% and 10% by weight of each of the two fibers being studied; the AFB1 was assayed as described above in Example 8.

The results which were obtained are depicted in the attached FIG. 8, in which the AFB1 adsorption, expressed as percentage reduction of the concentration of AFB1 in the supernatant as compared with the concentration initially present, depends on the quantity of fibers employed (in % by weight); the black squares correspond to the micronized wheat fibers while the black diamonds correspond to the nonmicronized wheat fibers.

These results show that the micronized wheat fibers are markedly more efficient with regard to absorbing the AFB1.

From the commercial point of view, it is interesting to note that the 0.75% dose of micronized wheat fibers has the same effect as the 5% dose of the same, nonmicronized fiber. At pH 3, both these quantities absorb 50% of the AFB1 in the model medium, which was initially contaminated with approximately 8 ppb. Consequently, the use of micronized plant fibers is of great commercial interest insofar as it makes it possible to decrease the quantity of raw material which is required for adsorbing a given quantity of microtoxins.

### EXAMPLE 10

### Confirmation of the Adsorption of OTA During a Pilot Brewing Process

1) Experimental Protocol

3 mashtubs were prepared in a pilot brewery, i.e. one control mashtub and two mashtubs to which Adfimax® BW was added to a dose of 10% by weight base on the malt.

The experimental conditions are summarized in Table VIII below:

TABLE VIII

| | Control | Mashtub A1 (with | Mashtub A2 (with |
|---|---|---|---|
| | mashtub | Adfimax ® BW) | Adfimax ® BW) |
| Quantity of malt (kg) | 40 | 40 | 40 |
| Quantity of Adfimax ® | 0 | 4 | 4 |
| BW (kg) | | | |
| Quantity of water | 140 | 140 | 140 |
| (in liters) | | | |

2) Results

The results which were obtained are presented in Table IX below:

TABLE IX

| | Control mashtub | Mashtub A1 | Mashtub A2 |
|---|---|---|---|
| Total quantity of | 8.66 | 1.44 | 1.96 |
| OTA in the beer | | | |
| (μg/batch) | | | |

A reduction of approximately 80% in the total contamination with OTA is therefore observed in the final beer.

### EXAMPLE 11

### Demonstration of the Adsorption of the OTA on Grape Fibers in a Model Medium

1) Experimental Protocol

The contaminated synthetic model medium DYP was previously treated in the same manner as in Example 1 above. The DYP was contaminated with OTA to a value of approximately 45 ng/ml.

Two types of micronized grape fibers were tested in this example: micronized grape pip fibers sold under the trade name Adfimax® 64 and micronized grape pulp fibers sold under the trade name Adfimax® 59.

The fibers employed were introduced into the contaminated medium at the rate of 20 g/l.

In the case of each of the two fibers tested, the experiment was performed at pH 6.3 (normal DYP medium) and at a pH of 4.5, corresponding to the normal DYP medium whose pH was adjusted to 4.5 with lactic acid.

The time for which the contaminated medium was in contact with the fibers was 45 minutes.

The residual quantity of OTA in the medium was determined as indicated above in Example 1.

2) Results

The results relating to the percentage adsorption of OTA are reported in Table X below:

TABLE X

| Nature of fibers | Adsorption of | Adsorption of OTA |
|---|---|---|
| employed | OTA at pH 6.3 (in %) | at pH 4.5 (in %) |
| Grape pip fibers | 7 | 30 |
| Grape pulp fibers | 35 | 68 |

These results show that the percentage adsorption by the grape pulp fibers vis-à-vis the OTA is very high, something which is very interesting from the commercial point of view since the process according to the invention can be applied to decontaminating wine or grape juice of microtoxins.

1. A biological process for decontaminating mycotoxins in a liquid dietary medium, characterized in that it comprises at least the following steps:
adsorbing at least a part of the mycotoxins, which are likely to be present in the liquid dietary medium to be decontaminated, by bringing said medium into contact with micronized insoluble plant fibers, and
removing said fibers on which the mycotoxins are absorbed.

2. The process as claimed in claim 1, characterized in that the insoluble plant fibers are fibers derived from:
dietary plants selected from cereals, leguminosae, culinary plants and fruits including tropical fruits;
plants derived from the paper industry and selected from trees, sugarcane, bamboo and cereal straw.

3. The process as claimed in claim 2, characterized in that the fibers derived from dietary plants are fibers derived from cereals and are selected from wheat, barley, oat, corn, millet, rice, rye and sorghum fibers and their malted equivalents.

4. The process as claimed in claim 2, characterized in that the insoluble plant fibers are selected from fibers derived from apples, pears, grape berries, lupin and soya bean seeds, tomatoes, peas and coffee.

5. The process as claimed in claim 1, characterized in that the fibers are present in the form of microparticles at least 90% of the total mass of which has a size of less than or equal to 700 μm.

6. The process as claimed in claim 5, characterized in that the fibers are present in the form of microparticles at least 90% of the total mass of which has a size of less than or equal to 200 μm.

7. The process as claimed in claim 1, characterized in that it additionally comprises a preliminary step during which the fibers are hydrated.

8. The process as claimed in claim 1, characterized in that the quantity of plant fibers introduced into the liquid medium to be decontaminated is between 0.1 and 20% by weight per liter of medium.

9. The process as claimed in claim 1, characterized in that the dietary medium is brought into contact with the plant fibers for a period of between a few seconds and 90 minutes.

10. The process as claimed in claim 1, characterized in that the dietary medium is brought into contact with the plant fibers at a pH of between 1.5 and 7.

11. The process as claimed in claim 1, characterized in that the medium is maintained at a temperature of between 7 and 80° C. during the whole of the period of contact.

12. The process as claimed in claim 1, characterized in that the medium is selected from beer, mixtures of malt and water and the mash of the brewing processes, wine, coffee, fruit juices, milk and glucose syrups.

13. The process as claimed in claim 1, characterized in that the fibers are removed by filtration at the end of the period of contact.

14. The process as claimed in claim 1, characterized in that the steps of bringing the liquid dietary medium to be decontaminated into contact with the insoluble plant fibers, on the one hand, and, on the other hand, of removing said fibers on which the mycotoxins are adsorbed are carried out simultaneously.

15. The process as claimed in claim 14, characterized in that the step of removing the fibers is a step of filtration and in that the insoluble plant fibers form an integral part of a filtration system.

16. The use of the process as defined in claim 1, for detoxifying the beer during a brewing process, with said brewing process involving at least one filtration operation.

17. A brewing process comprising at least one step of mashing and at least one step of fermenting a wort, characterized in that it additionally comprises at least one step of mycotoxin decontamination using the process as defined in claim 1, with said decontamination step taking place simultaneously with the mashing step and/or after the step of fermenting and/or maturing the wort.

18. The process as claimed in claim 17, characterized in that the decontamination step is carried out simultaneously with the mashing step by bringing a mixture of ground malt and water into contact with insoluble plant fibers, with said fibers on which the mycotoxins are then adsorbed being removed by the step of filtering the mash at the end of the mashing.

19. The process as claimed in claim 18, characterized in that the plant fibers are introduced at the rate of from 0.5 to 20% by weight based on the weight of malt.

20. The process as claimed in claim 13, characterized in that the step of bringing the liquid medium to be decontaminated into contact is carried out before the step of filtering a wort which is fermented and, where appropriate, matured, by bringing this wort into contact with insoluble plant fibers, with said fibers on which the mycotoxins are then adsorbed being removed by the step of filtering the fermented wort.

21. The process as claimed in claim 20, characterized in that the plant fibers are introduced into the fermented wort at the rate of from 0.05 to 5% by weight based on the total weight of the wort.
